(19) United States
(12) Patent Application Publication
(10) Pub. No.: US 2016/0051313 A1
(43) Pub. Date: Feb. 25, 2016

(54) Attachment for Electrosurgical System

(71) Applicants: Jerome Canady, Lakeland, FL (US); Taisen Zhuang, Rockville, MD (US)

(72) Inventors: Jerome Canady, Lakeland, FL (US); Taisen Zhuang, Rockville, MD (US)

(21) Appl. No.: 14/465,867

(22) Filed: Aug. 22, 2014

(51) Int. Cl. A61B 18/14; A61B 18/12

(52) U.S. Cl. CPC A61B 18/14; A61B 18/1206; A61B 2018/00601

(57) ABSTRACT

An electrosurgical scalpel for a gas-assisted electrosurgical system. The electrosurgical scalpel has a dielectric portion and a conductive portion. The dielectric portion forms a substantial majority of the outer surface of the scalpel such that the conductive portion is only exposed along a thin edge of the scalpel. The dielectric portion provided stiffness to the very thin conductive portion and causes energy to be concentrated at the thin edge of the scalpel.

### CROSS-REFERENCE TO RELATED APPLICATIONS

None.

### STATEMENT REGARDING FEDERALLY SPONSORED RESEARCH OR DEVELOPMENT

None.

### BACKGROUND OF THE INVENTION

1. Field of the Invention

The present invention relates to electrosurgical systems and methods, and more particularly, to electrodes for gas-assisted electrosurgical systems and methods.

2. Brief Description of the Related Art

The standard means for controlling traumatic and surgical blood loss are electrosurgical generators and lasers which respectively direct high-frequency electrical currents or light energy to localize heat in bleeding vessels so as to coagulate the overlying blood and vessel walls. Hemostasis and tissue destruction are of critical importance when removing abnormal tissue during surgery and therapeutic endoscopy. For monopolar electrosurgery electrical energy originates from an electrosurgical generator and is applied to target tissue via an active electrode that typically has a small cross-sectional surface-area to concentrate electrical energy at the surgical site. An inactive return electrode or patient plate that is large relative to the active electrode contacts the patient at a location remote from the surgical site to complete and electrical circuit through the tissue. For bipolar electrosurgery, a pair of active electrodes are used and electrical energy flows directly through the tissue between the two active electrodes.

U.S. Pat. No. 4,429,694 to McGreevy disclosed a variety of different electrosurgical effects that can be achieved depending primarily on the characteristics of the electrical energy delivered from the electrosurgical generator. The electrosurgical effects included pure cutting effect, a combined cutting and hemostasis effect, a fulguration effect and a desiccation effect. Fulguration and desiccation sometimes are referred to collectively as coagulation.

Another method of monopolar electrosurgery via argon plasma technology was described by Morrison in U.S. Pat. No. 4,040,426 in 1977 and by McGreevy in U.S. Pat. No. 4,781,175. This method, referred to as argon plasma coagulation (APC) or argon beam coagulation (ABC) is a non-contact monopolar thermoablative method of electrocoagulation that has been widely used in surgery for the last twenty years. In general, APC involves supplying an ionizable gas such as argon past the active electrode to target tissue and conducting electrical energy to the target tissue in ionized pathways as non-arcing diffuse current. Canady described in U.S. Pat. No. 5,207,675 the development of APC via a flexible catheter that allowed the use of APC in endoscopy. These new methods allowed the surgeon, endoscopist to combine standard monopolar electrocautery with a plasma gas for coagulation of tissue.

APC has been demonstrated to be effective in the coagulation of blood vessels and human tissue during surgery. APC functions in a noncontact manner. The electrical current is initiated only when the tip of the handpiece or catheter is within one centimeter of the target tissue and produces a homogenous 1 mm to 2 mm well-delineated eschar. The eschar created by APC is further characterized by a decrease absence of charring and carbonization compare to eschar resulting from conventional electrosurgical fulguration. The eschar remains firmly attached to the tissue, in contrast to other coagulation modalities where there is an overlying charred layer of coagulated blood. There is minimal tissue necrosis with APC.

In U.S. Pat. Nos. 5,217,457 and 5,088,997 to Delahuerga et al. disclosed a device for performing procedure referred to as “argon shrouded cut.” The device was an electrosurgical pencil having an exposed electrode with a distal end defining a tip for cutting biological tissue and a nose piece mounted about the electrode to define a pathway for a stream of inert gas which shrouds the electrode at or near its tip. When in coagulation mode, a convergent stream of inert gas was directed directly onto the tip of the electrode. In coagulation mode, the voltage was sufficient to initiate an electrical discharge in the inert gas. In cut mode, the stream of ionized gas was directed to impinge obliquely on the electrode at a point adjacent to but away from the tip of the electrode. In cutting mode, the open circuit voltage was generally not high enough to continuously plasmatize the inert gas and initiate and maintain an electrical discharge. Accordingly, in cut mode the function of the inert gas is to provide a shroud around the electrode rather than to initiate electrical discharge.

A multitude of literature exists that discloses and discusses various commercially available electrosurgical generators and the voltage waveforms produced by those generators. For example, A. Erwine, “ESU-2000 Series Product Overview A Paradigm Shift in Electrosurgery Testing Technology and Capability Is Here,” BC Group International, Inc. (2007) describes electrosurgical generators from ERBE Elektromedizin GmbH and ConMed Corporation, among others.

In U.S. Patent Application Publication No. US-2013-0296848, Canady et al. described electrosurgical systems and methods using argon plasma during cutting modes of operation. The disclosed electrosurgical device had a handpiece or pencil 100 having a rigid housing 110 and telescoping nozzle or tip 120. The rigid housing may be formed, for example, from molded sides 102 and 104. The two sides 102, 104 are joined to form housing 110 having a hollow chamber within. Within the housing 110 is a needle electrode 230, electrode tubing 270 and a fiberglass plate 240. The needle electrode 230 extends through the electrode tubing 270. The electrode tubing additional has within it a channel, tube or other means for conducting the inert gas from the distal end of tubing 220 through the electrode tubing 270 and out of the electrode tubing 270. The inert gas leaving the channel in the electrode tubing then passes out of an opening at the distal end of the nozzle 120. The fiberglass plate 240 and electrode 230 are connected to electrical cable assembly 210. The electrode tubing is connected at its distal end to the PVC hose tubing 220. An O-ring 260 is placed between the telescoping nozzle and the electrode tubing to form a seal there between. A ceramic tip 250 may be placed at a distal end of the telescoping tip or nozzle 120 to protect the nozzle 120 from heat damage where the electrode passes through an opening at the distal end of the nozzle 120. The electrical cable assembly extends from a proximal end of the housing 110 and has at its distal end a plug 212. During operation of the device, the connector 212 is connected to an electrosurgical generator. The PVC hose tubing 220 also extends from the proximal end of the housing 110 and has at its distal end a gas connector body 222, a gas connector tip 224 and an O-ring 226. During operation of the device, the gas connector assembly (222, 224, 226) is connected to a source of an inert gas such as argon. The housing 110 has a plurality of opening or holes for accommodating a plurality of controls or buttons 140, 150, 160. The telescoping nozzle or tip 120 has a control element 122 extending through a slot 112 in the housing 110. The control element, tab, know or slider 122 is used by a surgeon to move the telescoping tip 120 into or out of an opening in a distal end of the housing 120. Three controls or buttons 140, 150, 160, extend out of openings in the housing 110 and have springs 142, 152, 162 between them and fiberglass plate 240 to bias the controls or buttons away from the plate or connector 240.

The electrosurgical device of U.S. Patent Application Publication No. US-2013-0296848 could be operated, for example, in four different modes: conventional cut mode, conventional coagulation mode, argon plasma coagulation mode, and hybrid plasma cut mode. The eschar resulting from cutting and coagulation in the hybrid plasma cut mode in accordance with the present invention is substantially better than conventional fulguration, cutting and argon plasma coagulation techniques. In addition there is substantial absence of charring, carbonization, tissue necrosis and destruction of adjacent tissue. Thus, tissue can be precisely cut and the adjacent vessels simultaneously sealed with minimal depth of injury, tissue necrosis, eschar and carbonization.

Any generator that provides high-frequency voltage to ionize the inert gas to form a gas stream can be used. Preferred generators include the Canady Plasma™ Electrosurgery Unit model (SS-601 MCa) and the Canady Plasma™ Electrosurgery Unit model (SS-200E) that are preferably used with the Argon plasma units Canady Plasma™ Argon 4 Coagulator (CPC 4) and Canady Plasma™ Argon 2 Coagulator (CPC 2), respectively. The CPC 4 provides a controlled flow of inert gas to the electrosurgical device during argon plasma coagulation mode and in hybrid plasma cut mode. The flow rate and the power can be manually set. In a coagulation mode, the generator delivers, for example, a peak-to-peak voltage of less than 9000 volts. In a cut mode, for example, the generator delivers a peak-to-peak voltage of less than 3800 volts. Most preferably, a peak-to-peak voltage of 100 to 9000 volts is delivered by the generator. Any accessory devices could be attached to the electrosurgical unit/plasma unit combination. Exemplary devices are an electrosurgical device (a handpiece) or an argon plasma flexible probe (catheter), rigid or laparoscopic.

For operating the electrosurgical device disclosed in U.S. Patent Application Publication No. US-2013-0296848, high-frequency current can be activated by two push buttons for the conventional cut mode and the conventional coagulation mode, respectively. Argon gas may be delivered by activating a third push button. This activation will allow the argon plasma coagulation mode and the hybrid plasma cut mode. The plasma cut mode will cut and coagulate the tissue at the same time. It can be easily switched between the different modes by activating the respective buttons. The plasma or electrical current can also be activated by a footswitch.

In U.S. Patent Application Publication No. US-2013-0296848, the electrosurgical scalpel took the form of a needle or wire. Such a form is common with conventional electrosurgical staplers. Electrosurgical scalpels, however, can take many other forms, for example, as shown in U.S. Pat. No. 7,066,936 to Ryan, U.S. Pat. No. 6,610,057 to Ellman et al., U.S. Pat. No. 5,951,551 to Erlich,

### SUMMARY OF THE INVENTION

In a preferred embodiment, the present invention is an attachment for a gas-assisted electrosurgical device. The attachment comprises a housing, a channel within said housing, a connector for connecting said channel to a gas source, an electrosurgical scalpel having a width at least three times its thickness, said electrosurgical scalpel comprising and a connector for connecting said electrosurgical scalpel to an electrosurgical generator. The electrosurgical scalpel has a dielectric portion and a conductive portion. The dielectric provide stiffness to the electrosurgical scalpel. The conductive portion is exposed only along the thickness of said electrosurgical scalpel. The conductive portion of said electrosurgical scalpel may be a conductive plate having opposing flat surfaces formed by its width and length and an edge formed by its thickness. The dielectric portion may be a dielectric coating on said opposing flat surfaces of said conductive plate. The dielectric coating may form flat or contoured surfaces on the width of the scalpel. The electrosurgical attachment may further have a connector for connecting said scalpel to an electrosurgical handpiece.

In another embodiment, the dielectric portion is a dielectric plate or slab having a pair of opposing surfaces formed by its width and an edge formed by its thickness. The conductive portion of said electrosurgical scalpel comprises a conductive wire along said edge of said dielectric plate. The dielectric coating may have flat or contoured outer surfaces.

Still other aspects, features, and advantages of the present invention are readily apparent from the following detailed description, simply by illustrating a preferable embodiments and implementations. The present invention is also capable of other and different embodiments and its several details can be modified in various obvious respects, all without departing from the spirit and scope of the present invention. Accordingly, the drawings and descriptions are to be regarded as illustrative in nature, and not as restrictive. Additional objects and advantages of the invention will be set forth in part in the description which follows and in part will be obvious from the description, or may be learned by practice of the invention.

### BRIEF DESCRIPTION OF THE DRAWINGS

For a more complete understanding of the present invention and the advantages thereof, reference is now made to the following description and the accompanying drawings, in which:

FIG. 1 is a perspective view of a prior art electrosurgical handpiece having its electrode refracted within its housing.

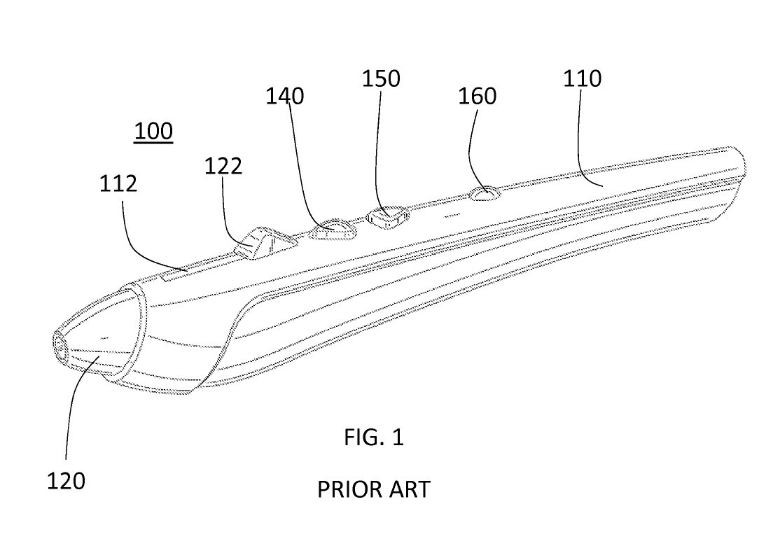

FIG. 2A is a perspective view of a prior art electrosurgical attachment having its electrode extending out from a distal end of its housing.

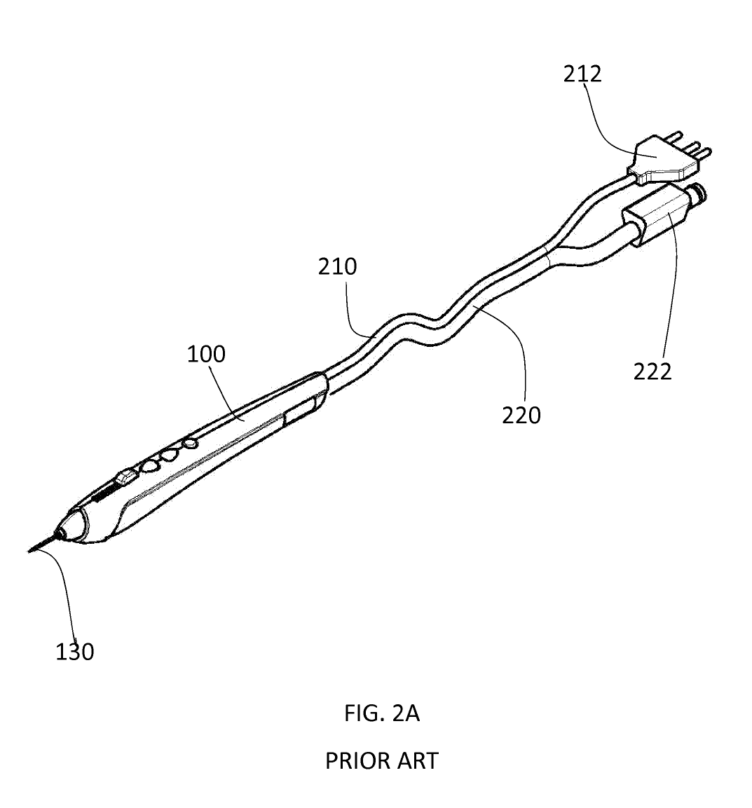

FIG. 2B is an assembly drawing of a prior art electrosurgical attachment of FIG. 2A.

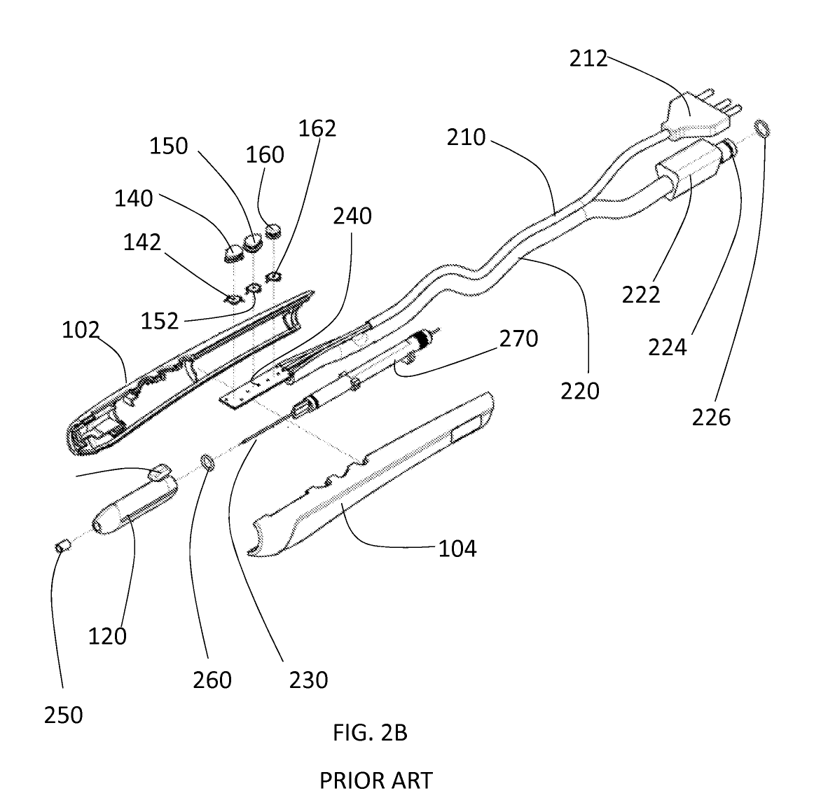

FIG. 3A is a perspective view of an electrode for an electrosurgical handpiece in an extended position in accordance with a first embodiment of the present invention.

FIG. 3B is a close-up perspective view of the electrode of FIG. 3A for an electrosurgical handpiece in an extended position in accordance with a first embodiment of the present invention.

FIG. 3C is a perspective view of an electrode for an electrosurgical handpiece in a retracted position in accordance with a first embodiment of the present invention.

FIG. 3D is a close-up perspective view of the electrode of FIG. 3C for an electrosurgical handpiece in a retracted position in accordance with a first embodiment of the present invention.

FIG. 4A is a perspective view of an electrode for an electrosurgical handpiece in an extended position in accordance with a second embodiment of the present invention.

FIG. 4B is a close-up perspective view of the electrode of FIG. 4A for an electrosurgical handpiece in an extended position in accordance with a second embodiment of the present invention.

FIG. 4C is a perspective view of an electrode for an electrosurgical handpiece in a retracted position in accordance with a second embodiment of the present invention.

FIG. 4D is a close-up perspective view of the electrode of FIG. 4C for an electrosurgical handpiece in a retracted position in accordance with a second embodiment of the present invention.

FIG. 5A is a perspective view of an electrode for an electrosurgical handpiece in an extended position in accordance with a third embodiment of the present invention.

FIG. 5B is a close-up perspective view of the electrode of FIG. 5A for an electrosurgical handpiece in an extended position in accordance with a third embodiment of the present invention.

FIG. 5C is a perspective view of an electrode for an electrosurgical handpiece in a retracted position in accordance with a third embodiment of the present invention.

FIG. 5D is a close-up perspective view of the electrode of FIG. 5C for an electrosurgical handpiece in a retracted position in accordance with a third embodiment of the present invention.

### DETAILED DESCRIPTION OF THE PREFERRED EMBODIMENTS

In the context of a hybrid plasma cut system such as is disclosed in U.S. Patent Application Publication No. US-2013-0296848, it has been found that decreasing the exposed surface area of the electrode, and in particular the cutting edge of the electrode, improves the quality of simultaneous cutting and coagulation. By making the electrosurgical scalpel thinner, such as in the form of a thin plate rather than a needle or wire, improves the quality of the cutting and coagulation. Making the scalpel thinner, however, reduces the stability of the electrode and can result in bending of the electrode. In the present invention, a scalpel is formed using a combination of conductive and dielectric material to produce a scalpel of sufficient strength yet limiting the exposed area of the conductive material to achieve improved cutting and coagulation.

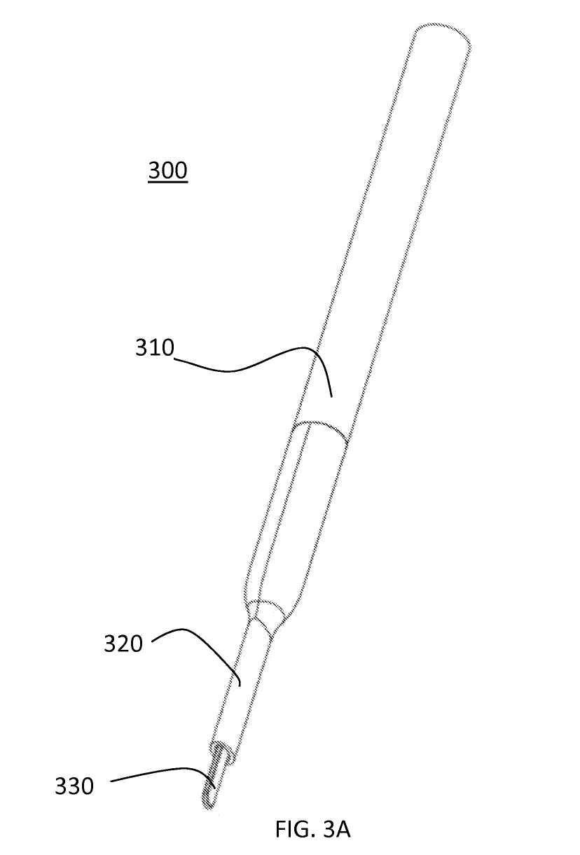

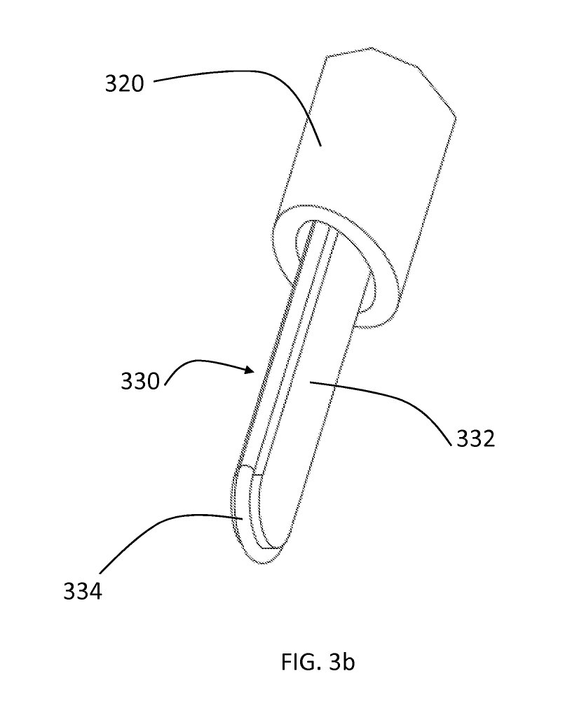

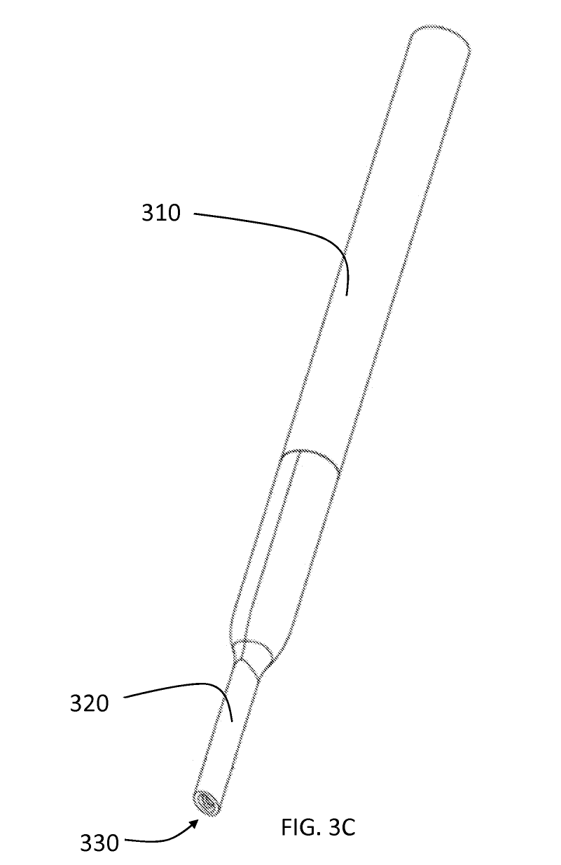

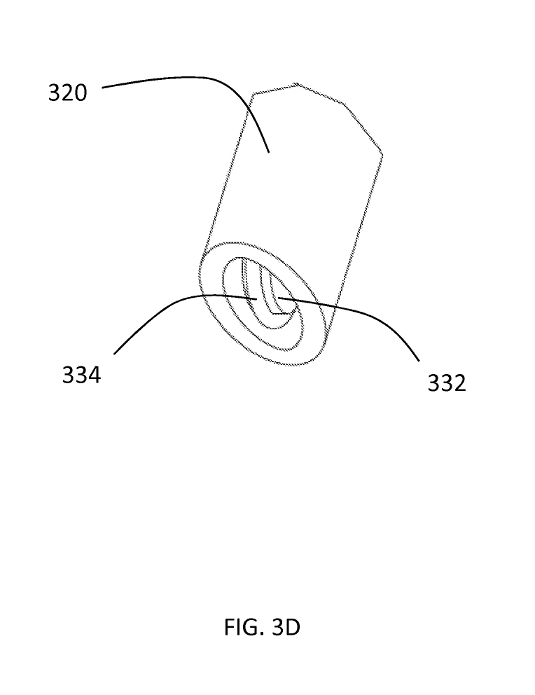

A first preferred embodiment of the present invention is shown in FIGS. 3A-3D. An electrosurgical handpiece 300 has a housing 310 formed from a rigid material such as plastic or other material known to those of ordinary skill in the art. A nozzle 320 extends from the housing 310 and may be integral with the housing 310 or may be attached to the housing. The housing 310 and the nozzle 320 having a channel therein for conducting gas from a gas source (not shown), through the housing and out a port at the distal end of the nozzle 320. An electrosurgical scalpel 330 is movably mounted in the nozzle and/or housing such that is can extend out of the nozzle 320 as shown in FIGS. 3A and 3B or can be retracted within the housing as shown in FIGS. 3C and 3D. As shown in FIG. 3B, the scalpel 330 is formed from a dielectric slab or plate 332, which provides rigidity to the scalpel. A thin wire 334, for example, 1 mm or less in diameter, is mounted around the edge of the dielectric slab or plate 332. With this structure, the wire 334 forms the cutting surface and electrode of the scalpel. The proximal end of the scalpel is connected to a source of electrical current such as an electrosurgical generator. Many different means are known in the art for making that connection.

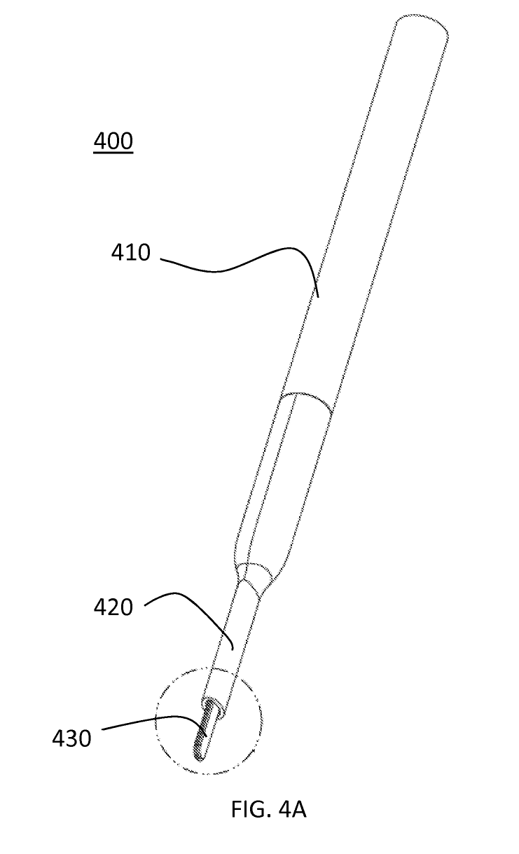

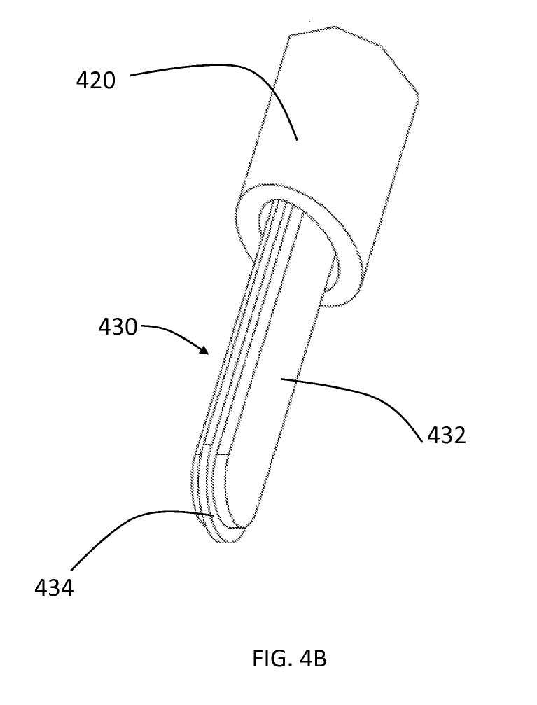

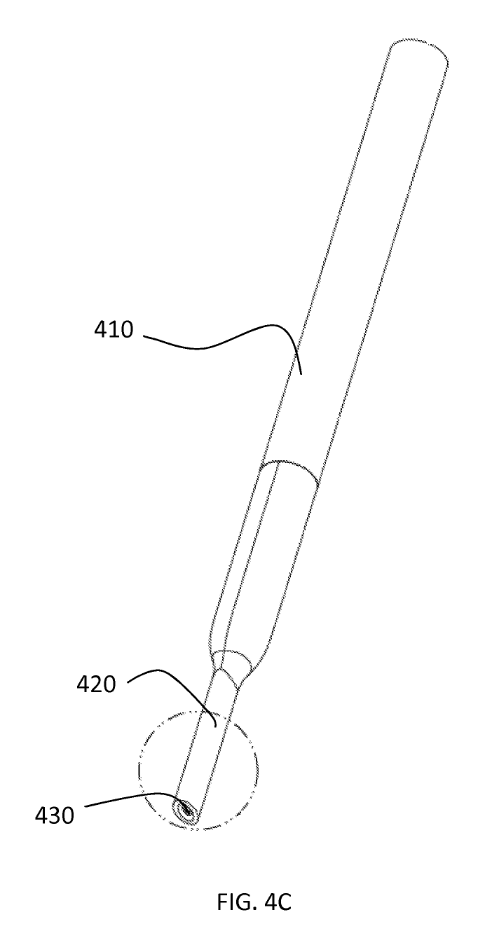

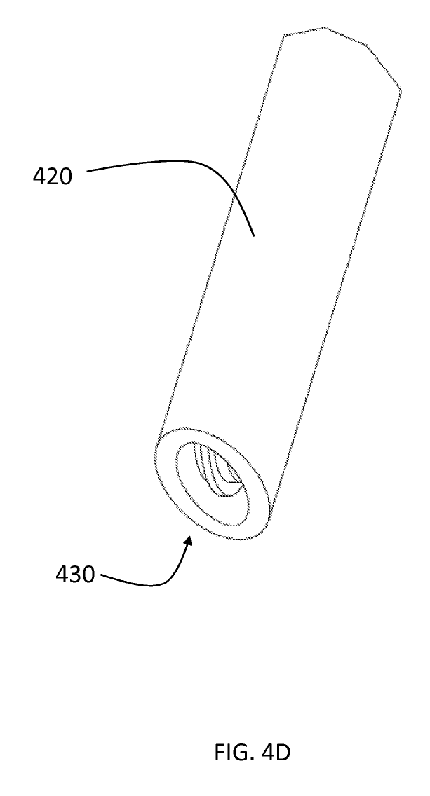

A second preferred embodiment of the present invention is shown in FIGS. 4A-4D. An electrosurgical handpiece 400 has a housing 410 formed from a rigid material such as plastic or other material known to those of ordinary skill in the art. A nozzle 420 extends from the housing 410 and may be integral with the housing 410 or may be attached to the housing. The housing 410 and the nozzle 420 having a channel therein for conducting gas from a gas source (not shown), through the housing and out a port at the distal end of the nozzle 420. An electrosurgical scalpel 430 is movably mounted in the nozzle and/or housing such that is can extend out of the nozzle 420 as shown in FIGS. 4A and 4B or can be retracted within the housing as shown in FIGS. 4C and 4D. As shown in FIG. 4B, the scalpel 430 is formed from conductive electrical plate 434 coated on both sides with a dielectric 434, which provides rigidity to the scalpel 430. With this structure, the exposed surface of the conductive plate 434 forms the cutting surface and electrode of the scalpel. The proximal end of the scalpel is connected to a source of electrical current such as an electrosurgical generator. Many different means are known in the art for making that connection.

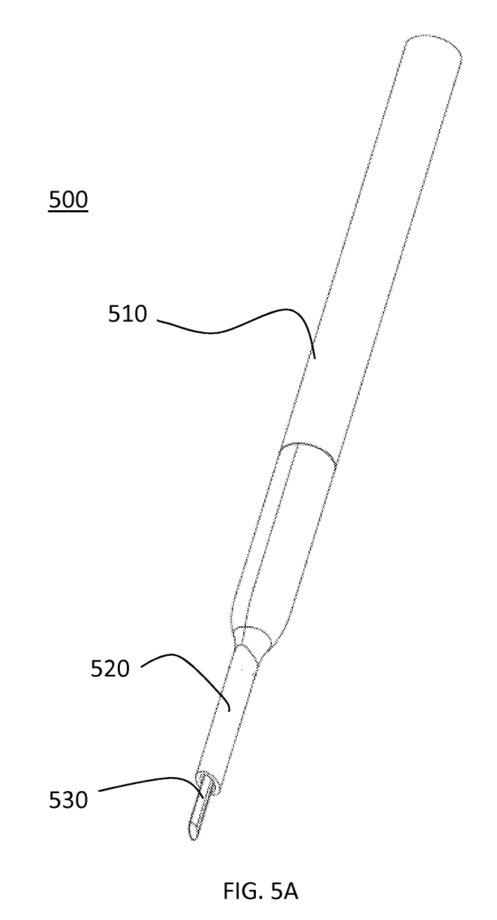

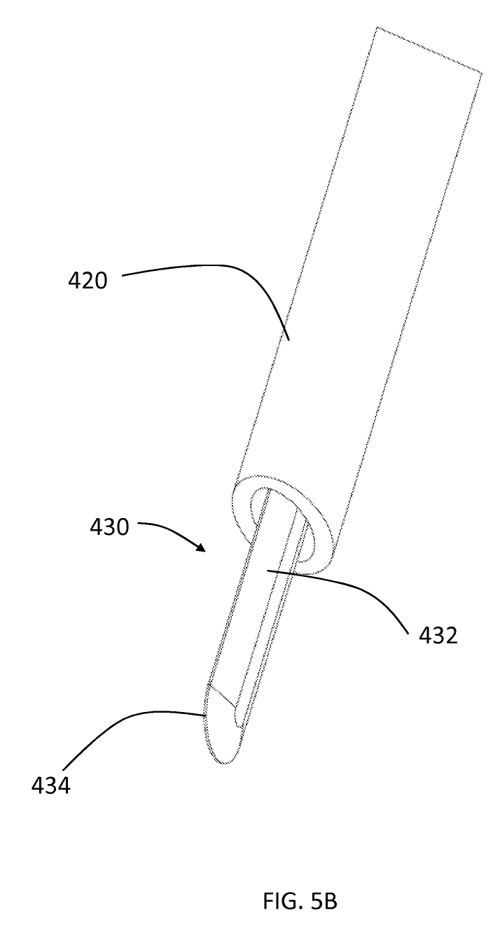

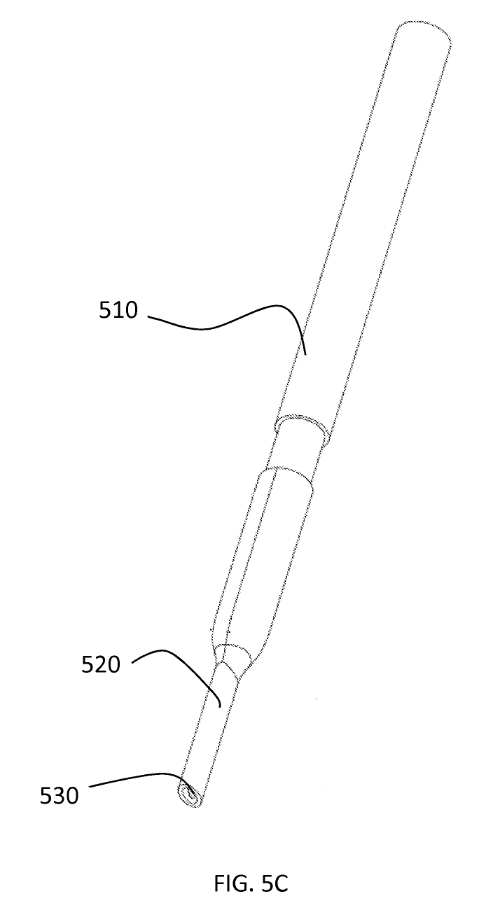

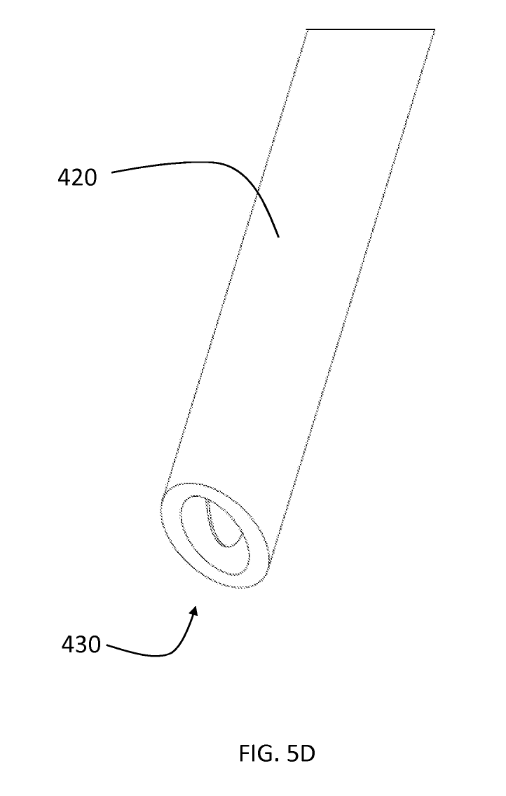

A third preferred embodiment of the present invention is shown in FIGS. 5A-5D. An electrosurgical handpiece 500 has a housing 510 formed from a rigid material such as plastic or other material known to those of ordinary skill in the art. A nozzle 520 extends from the housing 510 and may be integral with the housing 510 or may be attached to the housing 510. The housing 510 and the nozzle 520 having a channel therein for conducting gas from a gas source (not shown), through the housing and out a port at the distal end of the nozzle 520. An electrosurgical scalpel 530 is movably mounted in the nozzle and/or housing such that is can extend out of the nozzle 520 as shown in FIGS. 5A and 5B or can be retracted within the housing as shown in FIGS. 5C and 5D. As shown in FIG. 5B, the scalpel 530 is formed from conductive electrical plate 534. The conductive plate 534 is coated or covered with a dielectric 534, which provides rigidity to the scalpel 530. The dielectric 534 is then removed from the cutting edge of the conductive plate 534, for example, by sanding the edge. With this structure, the exposed surface of the conductive plate 534 forms the cutting surface and electrode of the scalpel. The proximal end of the scalpel is connected to a source of electrical current such as an electrosurgical generator. Many different means are known in the art for making that connection.

The foregoing description of the preferred embodiment of the invention has been presented for purposes of illustration and description. It is not intended to be exhaustive or to limit the invention to the precise form disclosed, and modifications and variations are possible in light of the above teachings or may be acquired from practice of the invention. The embodiment was chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. An attachment for a gas-assisted electrosurgical device comprising:
a housing;
a channel;
a connector for connecting said channel to a gas source;
an electrosurgical scalpel having a width at least three times its thickness, said electrosurgical scalpel comprising:
a dielectric portion; and
a conductive portion;
wherein said conductive portion is exposed only along the thickness of said electrosurgical scalpel; and
a connector for connecting said electrosurgical scalpel to an electrosurgical generator.

2. An attachment for a gas-assisted electrosurgical device according to claim 1, wherein:
said conductive portion of said electrosurgical scalpel comprises a conductive plate having opposing flat surfaces formed by its width and length and an edge formed by its thickness; and
said dielectric portion comprises a dielectric coating on said opposing flat surfaces of said conductive plate.

3. An attachment for a gas-assisted electrosurgical device according to claim 2, wherein said dielectric coating forms a flat outer surface of the electrosurgical scalpel.

4. An attachment for a gas-assisted electrosurgical device according to claim 2, wherein said dielectric coating forms a contoured outer surface of the electrosurgical scalpel.

5. An attachment for a gas-assisted electrosurgical device according to claim 1, wherein:
said dielectric portion comprises a dielectric plate having a pair of opposing surfaces form by its width and an edge formed by its thickness; and
said conductive portion of said electrosurgical scalpel comprises a conductive wire along said edge of said dielectric plate.

6. An attachment for a gas-assisted electrosurgical device according to claim 5, wherein said dielectric coating forms a flat outer surface of the electrosurgical scalpel.

7. An attachment for a gas-assisted electrosurgical device according to claim 5, wherein said dielectric coating forms a contoured outer surface of the electrosurgical scalpel.

8. An attachment for a gas-assisted electrosurgical device comprising:
an electrosurgical scalpel having a width at least three times its thickness, said electrosurgical scalpel comprising:
a dielectric portion; and
a conductive portion;
wherein said conductive portion is exposed only along the thickness of said electrosurgical scalpel.

9. An attachment for a gas-assisted electrosurgical device according to claim 8, wherein:
said conductive portion of said electrosurgical scalpel comprises a conductive plate having opposing flat surfaces formed by its width and length and an edge formed by its thickness; and
said dielectric portion comprises a dielectric coating on said opposing flat surfaces of said conductive plate.

10. An attachment for a gas-assisted electrosurgical device according to claim 9, wherein said dielectric coating forms a flat outer surface of the electrosurgical scalpel.

11. An attachment for a gas-assisted electrosurgical device according to claim 9, wherein said dielectric coating forms a contoured outer surface of the electrosurgical scalpel.

12. An attachment for a gas-assisted electrosurgical device according to claim 8, wherein:
said dielectric portion comprises a dielectric plate having a pair of opposing surfaces form by its width and an edge formed by its thickness; and
said conductive portion of said electrosurgical scalpel comprises a conductive wire along said edge of said dielectric plate.

13. An attachment for a gas-assisted electrosurgical device according to claim 12, wherein said dielectric coating forms a flat outer surface of the electrosurgical scalpel.

14. An attachment for a gas-assisted electrosurgical device according to claim 12, wherein said dielectric coating forms a contoured outer surface of the electrosurgical scalpel.

15. An attachment for a gas-assisted electrosurgical device according to claim 12, further comprising a connector for connecting said scalpel to an electrosurgical handpiece.
